(19) United States
(12) Patent Application Publication
(10) Pub. No.: US 2010/0274285 A1
(43) Pub. Date: Oct. 28, 2010

(54) ELASTOMERIC SPINAL IMPLANT WITH LIMIT ELEMENT

(75) Inventor: Jeffrey P. Rouleau, Maple Grove, MN (US)

Correspondence Address: MEDTRONIC;Attn: Noreen Johnson - IP Legal Department, 2600 Sofamor Danek Drive, MEMPHIS, TN, 38132, US

(73) Assignee: MEDTRONIC, INC., Minneapolis, MN (US)

(21) Appl. No.: 12/429,424

(22) Filed: Apr. 24, 2009

(51) Int. Cl. A61B 17/70; A61B 17/86

(52) U.S. Cl. 606/246; 606/301

(57) ABSTRACT

Embodiments of the invention may include a spinal implant that has both an elastomeric element for connecting between two anatomic structures and a limit element wrapped transversely around the elastomeric element along at least a portion of the elastomeric element. The limit element may be separately or in conjunction connected to the two anatomical structures.

### FIELD OF THE INVENTION

The present invention relates generally to the field of medical implants, and more particularly relates to a spinal implant with an elastomeric element and a limit element that limits the length to which the elastomeric element may be expanded.

### BACKGROUND

Various pathologies of the spine and other bony structures may be treated by preventing or severely limiting motion and allowing the structures to heal. Some pathologies may be treated by biasing structures in a particular direction. While existing rod and screw systems and plating systems provide limits to motion and provide stability, many of the systems prevent load transfer during fracture repair or spinal fusion. Failure to allow adequate stress across a fracture or spinal fusion area may prevent effective growth of bone because some bone grows, in part, in response to induced stresses. Pathologies, such as scoliosis, may be treated by pulling a spinal structure with a biasing force, such as a tether, on one or more convex sides of a spinal curvature to properly align the spinal structure. Additionally, other devices that provided stability in some directions, but allow movement in other directions, are embodied in sliding plates, “dynamic” devices, and “motion preserving” devices. Simple cords and cables have been used to limit motion in particular directions, similar to a natural ligament. However, tethers made from cords, cables, and the like may respond abruptly, and with great stress transfer to anchoring elements, when a tether becomes taught due to physiological loads. Abrupt loading can cause an anchoring element such as a screw to fracture or pull out of a bone in which the anchoring element is implanted.

There remains a need for implants that respond to applied forces with gradually increasing force over a relatively long zone of displacement without undergoing levels of strain that may damage materials from which the implants are made. Some improved implants may include a capacity to respond effectively to both tensile and compressive applied loads.

### SUMMARY

An embodiment of the invention is a spinal implant configured to connect between two vertebrae. Spinal implant embodiments may include a first bone screw, a second bone screw, and an elastomeric element with a first end, a second end, and a central section between the first end and the second end. The first end may be laterally expanded and include a hole therethrough to couple with the first bone screw, and the second end may be laterally expanded and include a hole therethrough to couple with the second bone screw. Spinal implant embodiments may also include a limit element wrapped transversely around the elastomeric element along at least a portion of the central section of the elastomeric element. The limit element may be connected to the first bone screw and be connected to the second bone screw.

Another embodiment of the invention is a spinal implant configured to connect between two vertebrae that may include a first bone screw, a second bone screw, and an elastomeric element with a first end coupled to the first bone screw, a second end coupled to the second bone screw, and a central section between the first end and the second end. The spinal implant may also include a continuous limit element that wraps around the first bone screw, wraps transversely around the elastomeric element along at least a portion of the central section of the elastomeric element, and wraps around the second bone screw.

Yet another embodiment of the invention is a spinal implant configured to connect between two vertebrae that may include a first bone screw, a second bone screw, and an elastomeric element with a first end, a second end, a central section between the first end and the second end, and a connection means for connecting with the first bone screw and the second bone screw. Embodiments may also include a limit means wrapped transversely around the central section and connected to the first end and the second end for restricting the length to which the elastomeric element may be expanded.

### BRIEF DESCRIPTION OF THE DRAWINGS

FIG. 1 is an elevation view of an embodiment of a spinal implant in a portion of a lumbar region of a spine.

FIG. 2 is a partially exploded perspective view of an embodiment of a spinal implant with portions of the spinal implant placed in portions of a spine.

FIG. 3 is a perspective view of a portion of a spinal implant of FIG. 2.

FIG. 4 is a front elevation view of the spinal implant of FIG. 3.

FIG. 5 is a side elevation view of the spinal implant of FIG. 3 with some of the components separated to illustrate various aspects of the spinal implant.

FIG. 6 is a perspective view of an embodiment of a spinal implant.

FIG. 7 is a perspective view of an embodiment of a spinal implant.

### DETAILED DESCRIPTION

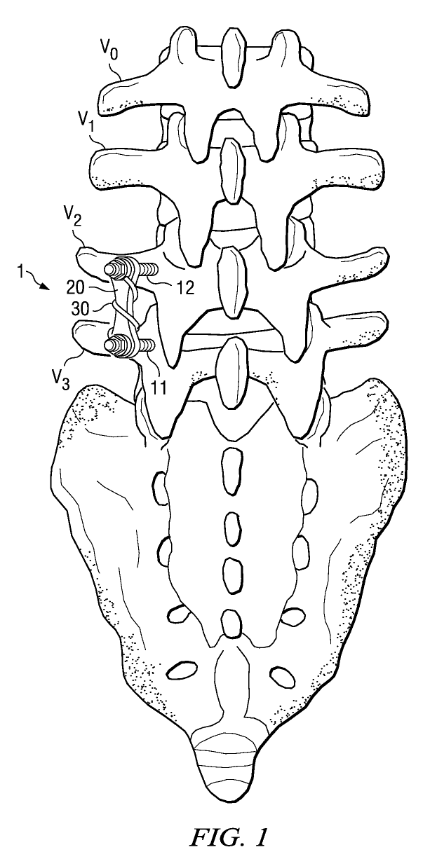

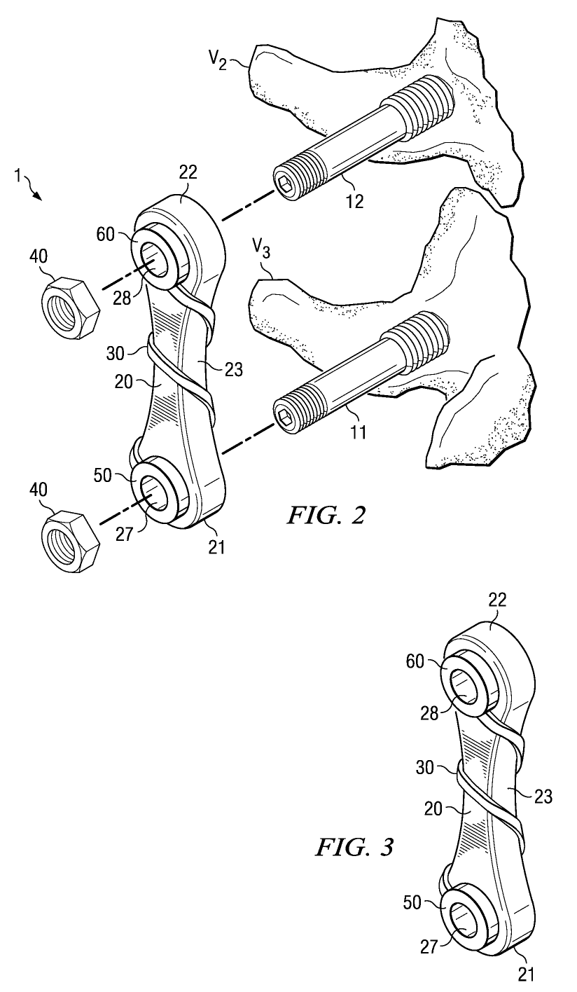

A spinal implant 1 connected between two vertebrae V₂, V₃ is illustrated in FIG. 1. Four vertebral bodies V₀, V₁, V₂, and V₃ are shown with the spinal implant 1 attached to the vertebral bodies V₂ and V₃. The spinal implant 1 includes an elastomeric element 20 and a limit element 30 attached to the vertebral bodies V₃ and V₂ with a first bone screw 11 and a second bone screw 12. The first and second bone screws 11, 12 may be of any effective type capable of connecting to bone. The first and second bone screws 11, 12 may have any functional thread pattern or driving mechanism, or be configured to connect any implant, such as but not limited to, an orthopedic plate, a spinal plate, an interbody device, or an extradiscal device. In the illustrated embodiment, the bone screws 11, 12 are pedicle screws with posts to which nuts 40 (FIG. 2) may be attached to secure the elastomeric element 20 to the bone screws 11, 12.

An embodiment of the spinal implant 1 configured to connect between two vertebrae is illustrated in FIGS. 2-5. In FIG. 2, the first bone screw 11 is shown partially implanted in the vertebra V₃. The second bone screw 12 is shown partially implanted in the vertebra V₂. An elastomeric element 20 with a first end 21, a second end 22, and a central section 23 between the first end 21 and the second end 22 is configured to couple with the first bone screw 11 and the second bone screw 12. In the illustrated embodiment, the first end 21 is laterally expanded and includes a hole 27 through the elastomeric element 20. The illustrated second end 22 is also laterally expanded and includes a hole 28 through the elastomeric element 20. In various embodiments, connection to an elastomeric element may be by other devices and the first and second ends 21, 22 may or may not be laterally expanded.

The elastomeric element 20 of some embodiments is designed to provide a relatively compliant but consistent resistive force to separation of the first end 21 from the second end 22. In some embodiments, the resistance provided by deformation of the elastomeric element 20 is resigned to remain within a range that does not permanently stretch or deform the elastomeric element 20. The elastomeric element 20 may be made from any biocompatible material. However, certain more elastic materials and arrangements of material may be advantageous for some embodiments. The elastomeric element 20 may include a solid, elastic material and may in whole or in part be woven, knitted, or braided from strands or other smaller component parts. The elastomeric element 20 may be formed from elastic, inelastic, semi-elastic material, or some combination of these or other materials. Non-limiting example materials from which the elastomeric element 20 may be made include silicone, silicone-polyurethane, polyurethane, polyolefin rubbers, hydrogels, Nitinol or other superelastic alloys, and a fluid contained within a vessel. Materials which may be used for strands, fibers, or other components of the elastomeric element 20 include but are not limited to titanium, memory-wire, ultra-high molecular weight polyethylene (UHMWPE), cross-linked UHMWPE, polytetrafluoroethylene (PTFE), ethylene chlorotetrafluoroethylene, polyetheretherketone (PEEK), polyetherketoneketone (PEKK), fluorinated ethylene-propylene, and polyvinyl flouride. Further, combinations of superelastic alloys and non-metal elastic materials may be suitable to form elastic strands used in the formation of embodiments of the elastomeric element 20.

The cross-sectional shape of the elastomeric element 20 illustrated in FIGS. 2-5 is substantially rectangular. The cross-sectional area near the central section 23 is smaller than the cross-sectional area closer to the holes 27, 28 where the elastomeric element 20 is laterally expanded to couple with the first and second bone screws 11, 12. In some embodiments, the cross-sectional area may be more equal along a length of the elastomeric element 20 or even larger near the central section 23 to achieve certain structural characteristics. For example, where a design objective is to provide an elastomeric element 20 that is more capable in compression, a larger cross-sectional area near the central section 23 may be provided. Any other functional cross-sectional shape may be used for the elastomeric element 20 at any point along the length of the elastomeric element 20. For example and without limitation, the cross-sectional shape of the elastomeric element 20 may be round, oval, triangular, square, any other polygonal shape, or a non-symmetrical shape that provides more resistance to loading in one direction than it does in another direction.

The elastomeric element 20 may be particularly designed to resist compressive loads in some embodiments. Such a design may be useful to maintain minimal vertebral spacing with some devices. In other words, an elastomeric element 20 in use with pedicle screws that resists a compressive load in, for example, a state of spinal extension would aid in preserving a minimal vertebral spacing at the posterior of a disc space. A particular compressive load resistance in combination with a maximum allowed spinal flexion, as describe herein in association with a limit element, may define an implant that mimics natural flexion-extension characteristics of a healthy spine.

The limit element 30 is shown in FIGS. 2-5 wrapped transversely around the elastomeric element 20, including some of the limit element 30 wrapped along a portion of the central section 23. By “wrapped transversely” as used herein, it is meant that the limit element 30 wraps around a shorter dimension of the elastomeric element 20 that is transverse to a longitudinal axis of the elastomeric element 20. The limit element 30 may comprise a multi-strand cable, a wire, or a cord of any type. The limit element 30 may be solid, woven, knitted, braided, or any functional configuration. The illustrated limit element 30 is rectangular in cross-sectional shape. However, various embodiments of the limit element 30 may be of any functional shape.

The limit element 30 may be made from any biocompatible material noted above for use in making the elastomeric element. In some embodiments, the limit element 30 does not significantly stretch along its length under load, but is substantially inelastic. In other embodiments, the limit element 30 may have elastic characteristics. The elastic characteristics of the limit element 30 are considered in combination with characteristics of the elastomeric element 20 in designing a spinal implant 1 with required tensile and compressive characteristics. For example, the angle of placement of the limit element 30 around the elastomeric element 20, the number of times the limit element 30 is wrapped around the elastomeric element 20, the strength and elasticity of each element, and the cross-sectional shape of each element may be varied to achieve a spinal implant with particular characteristics. The number of revolutions may be chosen to meet design criteria and may include any functional number of revolutions. A larger number of revolutions will tend to increase the distance an implant will expand under tension. When tension is applied to a wrapped limit element 30, the limit element 30 will constrict around the elastomeric element 20 and continue to allow longitudinal expansion of the spinal implant 1 until the constricting force is balanced by resilience of the elastomeric element 20 against the limit element 30. With a larger number of revolutions, a longer limit element 30 will constrict against the elastomeric element 20 over a greater surface area, and therefore the elastomeric element 20 will not be deformed as greatly and respond with an opposite resilient force as quickly as would an implant with a smaller number of revolutions. A more compliant implant that results from a large number of revolutions may be particularly useful for a clinical situation such as treating a person with weaker bones, such as osteoporotic bones. A general purpose for a limit element may be to restrict spinal flexion to a selected maximum amount. Various designs further refine devices to not only define a spinal flexion limit, but to control the loads resisted at different amounts of flexion. The more compliant device mentioned above is an example of an implant designed to particularly control loads resisted based on deflections.

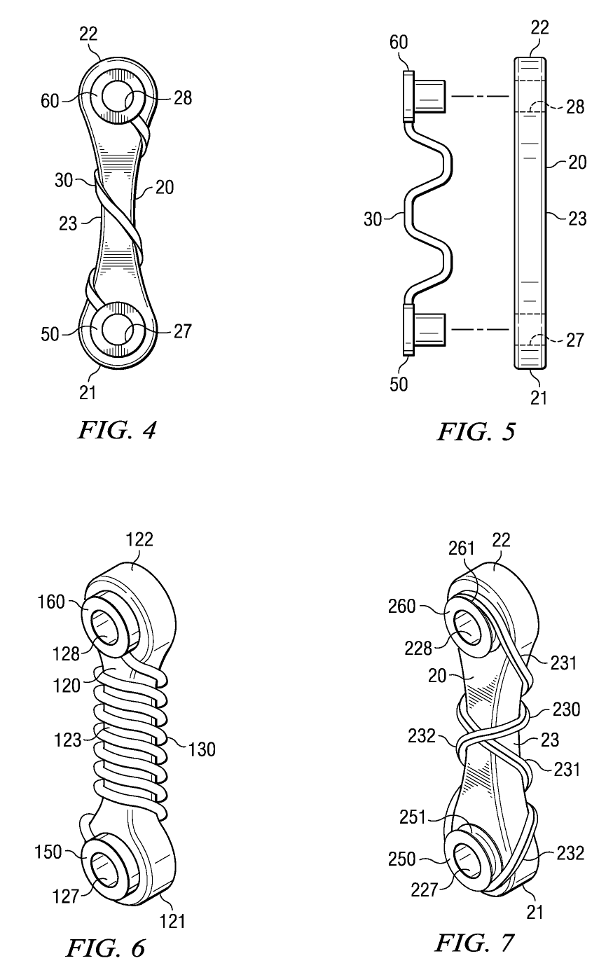

The spinal implant 1 illustrated in FIGS. 2-5 includes the limit element 30 wrapped transversely around the elastomeric element 20 for about two revolutions. The limit element 30 follows an approximately helical pattern around the elastomeric element 20. As can be seen clearly in FIG. 4, the limit element 30 extends from a first grommet 50 and a second grommet 60 at substantially equivalent angles and each is offset slightly from the central longitudinal axis of the elastomeric element 20 to reduce eccentric loads induced at the joints between the limit element 30 and the first and second grommets 50, 60 when a tensile load is applied between the first and second grommets 50, 60. FIG. 5 shows the limit element 30 and the first and second grommets 50, 60 separated from the elastomeric element 20. In this view, a profile of the limit element 30 illustrates the path of the limit element 30, and the size and orientation of the holes 27, 28, as well as the first and second grommets 50, 60, are clearly shown. In this embodiment, the limit element 30 is connected to the first bone screw 11 and to the second bone screw 12 respectively through the first grommet 50 and the second grommet 60. In other embodiments, connections between the limit element 30 and the first and second bone screws 11, 12 may be direct or the connections may be made through other components. For example and without limitation, connection to a bone screw may be made by wrapping the limit element 30 around a component that intercedes between the bone screw and the limit element 30, or connection may include contact between a limit element 30 that is wrapped around one or both of the bone screws 11, 12.

The illustrated first and second grommets 50, 60 extend through the elastomeric element 20 respectively at the holes 27, 28. The first and second grommets 50, 60 provide bearing surfaces between the elastomeric element 20 and the first and second bone screws 11, 12. The first and second grommets 50, 60 provide a larger surface area for bearing against the first and second bone screws 11, 12, and the first and second grommets 50, 60 may protect the material of the elastomeric element 20 from the potentially hard and sharp components of the first and second bone screws 11, 12. The limit element 30 illustrated in FIGS. 2-5 is connected directly to the first and second grommets 50, 60. This connection mechanism may be through any functional device. For example and without limitation, the connection mechanism between a grommet and the limit element 30 may be by a clamp, by welding, with adhesive, by wrapping some or all of the limit element 30 around a grommet, or by weaving or otherwise integrating some or all of the limit element 30 into a grommet.

Another embodiment of a spinal implant is illustrated in FIG. 6. An elastomeric element 120 is designed to provide a relatively compliant but consistent resistive force to separation of the first end 121 from the second end 122. The elastomeric element 120 may be made from any biocompatible material. However, certain more elastic materials and arrangements of material may be advantageous for some embodiments. Example materials and arrangements of materials of the elastomeric element 120 are essentially similar to those describe in association with the elastomeric element 20 herein.

The cross-sectional shape of the elastomeric element 120 illustrated in FIG. 6 is substantially circular over the length of most of the elastomeric element 120. The cross-sectional area near the central section 123 is essentially constant across a majority of the length of the elastomeric element 120, but the cross-sectional area is smaller than the cross-sectional area closer to the holes 127, 128 where the elastomeric element 120 is laterally expanded to couple with fasteners, such as the first and second bone screws 11, 12. The cross-sectional area of the elastomeric element 120 may be used as a design element to achieve certain structural characteristics, as noted in more detail in association with the elastomeric element 20 herein. For example, where a design objective is to provide an elastomeric element 120 that is more capable in compression, a larger cross-sectional area near the central section 123 may be provided. Any other functional cross-sectional shape may be used for the elastomeric element 120 at any point along the length of the elastomeric element 120. For example and without limitation, the cross-sectional shape of the elastomeric element 120 may be oval, rectangular, triangular, any other polygonal shape, or a non-symmetrical shape that provides more resistance to loading in one direction than it does in another direction.

The limit element 130 is shown in FIG. 6 wrapped transversely around the elastomeric element 120, including some of the limit element 130 wrapped along a portion of the central section 123. By “wrapped transversely” as used herein, it is meant that the limit element 130 wraps around a shorter dimension of the elastomeric element 120 that is transverse to a longitudinal axis of the elastomeric element 120. The limit element 130 may comprise a multi-strand cable, a wire, or a cord of any type. The limit element 130 may be solid, woven, knitted, braided, or any functional configuration. The illustrated limit element 130 is essentially circular in cross-sectional shape. However, various embodiments of the limit element 130 may be of any functional shape. Other physical and design characteristics and related variations of the limit element 130 are essentially similar to those of the limit element 30 described herein.

The limit element 130 illustrated in FIG. 6 is wrapped transversely around the elastomeric element 120 for about eight revolutions. The limit element 130 follows an approximately helical pattern around the elastomeric element 120. The limit element 130 extends from a first grommet 150 and a second grommet 160 at substantially equivalent angles and each is offset slightly from the central longitudinal axis of the elastomeric element 120 to reduce eccentric loads induced at the joints between the limit element 130 and the first and second grommets 150, 160 when a tensile load is applied between the first and second grommets 150, 160. In the embodiment of FIG. 6, the limit element 130 may be connected to bone screws or other fasteners through the first grommet 150 and the second grommet 160. In other embodiments, connections between the limit element 130 and fasteners may be direct or the connections may be made through other components.

The illustrated first and second grommets 150, 160 extend through the elastomeric element 120 respectively at the holes 127, 128. The first and second grommets 150, 160 provide bearing surfaces between the elastomeric element 120 and fasteners to which the elastomeric element 120 may be connected. The first and second grommets 150, 160 provide a larger surface area for bearing against fasteners, and the first and second grommets 150, 160 may protect the material of the elastomeric element 120 from potentially hard and sharp components of fasteners. The limit element 130 illustrated in FIG. 6 is connected directly to the first and second grommets 150, 160. This connection mechanism may be through any functional device. For example and without limitation, the connection mechanism between a grommet and the limit element 130 may be by a clamp, by welding, with adhesive, by wrapping some or all of the limit element 130 around a grommet, or by weaving or otherwise integrating some or all of the limit element 130 into a grommet.

Another embodiment of a spinal implant is illustrated in FIG. 7. The illustrated elastomeric element 20 is the same as the component described in FIGS. 2-5 and should be considered to include at least the same features and variations noted above. The limit element 230 is shown in FIG. 7 wrapped transversely around the elastomeric element 20, including some of the limit element 230 wrapped along a portion of the central section 23. By “wrapped transversely” as used herein, it is meant that the limit element 230 wraps around a shorter dimension of the elastomeric element 20 that is transverse to a longitudinal axis of the elastomeric element 20. The limit element 230 may comprise a multi-strand cable, a wire, or a cord of any type. The limit element 230 may be solid, woven, knitted, braided, or any functional configuration. The illustrated limit element 230 is essentially rectangular in cross-sectional shape. However, various embodiments of the limit element 230 may be of any functional shape. Other physical and design characteristics and related variations of the limit element 230 are essentially similar to those of the limit element 30 described herein.

The limit element 230 illustrated in FIG. 7 is wrapped transversely around the elastomeric element 20. The limit element 230 may more particularly be a continuous loop that wraps around first and second bone screws and the elastomeric element 20. For the purpose of the following discussion, assume that a fastener, such as the first bone screw 11 may be placed through a hole 227 in the first grommet 250. Additionally, assume that a fastener, such as the second bone screw 12 may be placed through a hole 228 in the second grommet 260. The limit element 230 wraps around the first bone screw 11 by wrapping around the first grommet 250 and passing over a shoulder 251 formed in the first grommet 250. The shoulder 251 may include a groove in which the limit element 230 will fit. Therefore, the shoulder 251 may help to guide and secure the limit element 230 to the first grommet 250. The limit element 230 may be secured with the shoulder 251 by any effective device. For example and without limitation, the limit element 230 may be fastened to the shoulder 251 with an adhesive, by melting a portion of either component, by welding, by interference fit, or by capturing either component within multiple other parts that can be fastened together. Each half of the continuous loop limit element 230 has been given a designation for discussion herein: first portion 231; and second portion 232. The second portion 232 is shown exiting from the shoulder 251 on the right side of the illustration in FIG. 7. The second portion 232 wraps transversely around the elastomeric element 20 along a portion of the central section 23. As viewed from the top of the elastomeric element 20, the second portion 232 of the limit element 230 wraps around the elastomeric element 20 in a counterclockwise direction as it progresses from the first end 21 to the second end 22. The second portion 232 of the limit element 230 wraps around the second bone screw 12 by wrapping around the second grommet 260 and passing over a shoulder 261 formed in the second grommet 260. The shoulder 261 may include a groove in which the limit element 230 will fit. Therefore, the shoulder 261 may help to guide and secure the limit element 230 to the second grommet 260. The limit element 230 may be secured with the shoulder 261 by any effective device, as more specifically detailed with regard to the shoulder 251 above. The limit element 230, further designated as the first portion 231 where it passes from the second grommet 260, wraps transversely around the elastomeric element 20 along a portion of the central section 23. As viewed from the top of the elastomeric element 20, the first portion 231 of the limit element 230 wraps around the elastomeric element 20 in a clockwise direction as it progresses from the second end 22 to the first end 21. In other embodiments, direction of overlapping wrappings of the elastomeric element 20 by the limit element 230 may be in the same rotational direction or may be in the same direction for a portion and in opposite directions for another portion.

The illustrated first and second grommets 250, 260 extend through the elastomeric element 20 respectively at the holes 227, 228. The first and second grommets 250, 260 provide bearing surfaces between the elastomeric element 20 and fasteners to which the elastomeric element 20 may be connected. The first and second grommets 250, 260 provide a larger surface area for bearing against fasteners, and the first and second grommets 250, 260 may protect the material of the elastomeric element 20 from potentially hard and sharp components of fasteners.

An embodiment of the invention is a spinal implant configured to connect between two vertebrae. The spinal implant may include a first bone screw, a second bone screw, an elastomeric element, and a limit means. The elastomeric element may have a first end, a second end, a central section between the first end and the second end. The elastomeric element may also include a connection means for connecting with the first bone screw and the second bone screw. The connection means may be of any effective type describe herein with regard to the elastomeric element, or an otherwise effective means. The spinal implant may also include a limit means wrapped transversely around the central section and connected to the first end and the second end for restricting the length to which the elastomeric element may be expanded. The limit means may be of any effective type describe herein with regard to the limit element, or an otherwise effective means.

In a method of manufacturing a spinal implant, a continuous limit element, such as the limit element 230, may be looped in an appropriate pattern with an opening through a central region of the loops. An elastomeric element 20 may then be passed into the central region such that the elastomeric element 20 fills the central region and is configured to capture portions of the limit element 230 near the first and second ends 21, 22 of the elastomeric element 20 by any mechanism described herein. In some embodiments, an act of the method may also be to flex the elastomeric element 20 to assist with capturing, or positioning for capture, portions of the limit element 230 near the first and second ends 21, 22.

Various method embodiments of the invention are described herein with reference to particular medical implants. However, in some circumstances, each disclosed method embodiment may be applicable to each of the medical implants, or to some other implant operable as disclosed with regard to the various method embodiments.

Terms such as front, side, lateral, longitudinal, top, bottom, posterior, and the like have been used herein to note relative positions. However, such terms are not limited to specific coordinate orientations, but are used to describe relative positions referencing particular embodiments. Such terms are not generally limiting to the scope of the claims made herein.

While embodiments of the invention have been illustrated and described in detail in the disclosure, the disclosure is to be considered as illustrative and not restrictive in character. All changes and modifications that come within the spirit of the invention are to be considered within the scope of the disclosure.

What is claimed is:

1. A spinal implant configured to connect between two vertebrae comprising:
a first bone screw;
a second bone screw;
an elastomeric element with a first end, a second end, and a central section between the first end and the second end, wherein the first end is laterally expanded and includes a hole therethrough to couple with the first bone screw and the second end is laterally expanded and includes a hole therethrough to couple with the second bone screw; and
a limit element wrapped transversely around the elastomeric element along at least a portion of the central section of the elastomeric element, and wherein the limit element is connected to the first bone screw and is connected to the second bone screw.

2. The spinal implant of claim 1 wherein the first bone screw is a pedicle screw.

3. The spinal implant of claim 1 wherein the second bone screw is a pedicle screw.

4. The spinal implant of claim 1 wherein the elastomeric element comprises silicone.

5. The spinal implant of claim 1 wherein the limit element comprises a multi-strand cable.

6. The spinal implant of claim 1 wherein the limit element is wrapped transversely around the elastomeric element between about two and about eight revolutions.

7. The spinal implant of claim 1 wherein the limit element wraps around the first bone screw.

8. The spinal implant of claim 1 wherein the limit element wraps around the second bone screw.

9. The spinal implant of claim 1 wherein the limit element is continuous and wraps around the first bone screw, wraps transversely around the elastomeric element along at least a portion of the central section of the elastomeric element from the first end to the second end, wraps around the second bone screw, and wraps transversely around the elastomeric element along at least a portion of the central section of the elastomeric element from the second end to the first end.

10. The spinal implant of claim 9 wherein the limit element wraps transversely around the elastomeric element along at least a portion of the central section of the elastomeric element from the first end to the second end in a counterclockwise direction and wraps transversely around the elastomeric element along at least a portion of the central section of the elastomeric element from the second end to the first end in a clockwise direction.

11. The spinal implant of claim 1, further comprising a first grommet that penetrates at least partially through the first end of the elastomeric element and provides a bearing surface between the elastomeric element and the first bone screw.

12. The spinal implant of claim 11 wherein the first grommet comprises a connection mechanism between the limit element and the first bone screw.

13. The spinal implant of claim 1, further comprising a second grommet that penetrates at least partially through the second end of the elastomeric element and provides a bearing surface between the elastomeric element and the second bone screw.

14. The spinal implant of claim 13 wherein the second grommet comprises a connection mechanism between the limit element and the second bone screw.

15. A spinal implant configured to connect between two vertebrae comprising:
a first bone screw;
a second bone screw;
an elastomeric element with a first end coupled to the first bone screw, a second end coupled to the second bone screw, and a central section between the first end and the second end; and
a continuous limit element that wraps around the first bone screw, wraps transversely around the elastomeric element along at least a portion of the central section of the elastomeric element, and wraps around the second bone screw.

16. The spinal implant of claim 15 wherein the continuous limit element wraps around the first bone screw, wraps transversely around the elastomeric element along at least a portion of the central section of the elastomeric element from the first end to the second end in a counterclockwise direction, wraps around the second bone screw, and wraps transversely around the elastomeric element along at least a portion of the central section of the elastomeric element from the second end to the first end in a clockwise direction.

17. A spinal implant configured to connect between two vertebrae comprising:
a first bone screw;
a second bone screw;
an elastomeric element with a first end, a second end, a central section between the first end and the second end, and a connection means for connecting with the first bone screw and the second bone screw; and
a limit means wrapped transversely around the central section and connected to the first end and the second end for restricting the length to which the elastomeric element may be expanded.

18. The spinal implant of claim 17 wherein the connection means comprises a grommet that penetrates at least partially through the elastomeric element and provides a bearing surface between the elastomeric element and at least one of the first bone screw and the second bone screw.

19. The spinal implant of claim 18 wherein the grommet includes a coupling means for coupling with the limit means.

20. The spinal implant of claim 18 wherein the grommet includes a shoulder over which at least a portion of the connection element extends.
